(19) United States
(12) Patent Application Publication
(10) Pub. No.: US 2020/0069750 A1
(43) Pub. Date: Mar. 5, 2020

(54) EXTRACTS FROM ARTHROSPIRA AND USES THEREOF

(71) Applicant: Biovite Australia Pty Ltd, West Burleigh (AU)

(72) Inventors: Leodevico L. Ilag, Brighton (AU); Peter Owen Johnston, Carrara (AU)

(21) Appl. No.: 16/614,271

(22) PCT Filed: May 23, 2018
(86) PCT No.: PCT/AU2018/050498

§ 371 Date: Nov. 15, 2019

(30) Foreign Application Priority Data
May 23, 2017 (AU) 2017901961

(51) Int. Cl. A61K 35/748; G01N 33/68; A61P 29/00; A61P 17/00

(52) U.S. Cl. CPC A61K 35/748; A61P 17/00; A61P 29/00; G01N 33/6869

(57) ABSTRACT

The present invention relates extracts from physiologically stressed Arthrospira having anti-inflammatory activity and/or the ability to stimulate growth of new cells, compositions comprising the extracts having anti-inflammatory activity and/or the ability to stimulate growth of new cells, and use of such compositions in the treatment of diseases or conditions having an inflammatory basis and/or in the treatment of diseases or conditions of skin, wherein the diseases or conditions of skin are not the result of a microbial infection or infestation.

### TECHNICAL FIELD

The present invention relates to biological extracts having anti-inflammatory activity and/or the ability to stimulate growth of new cells, compositions comprising a biological extract having anti-inflammatory activity and/or the ability to stimulate growth of new cells, and use of such compositions in the treatment of diseases or conditions having an inflammatory basis and/or in the treatment of diseases or conditions of skin.

### BACKGROUND ART

The interleukin-1 receptor (IL-1R) is a cytokine receptor that is a key mediator of inflammatory processes. IL-1R is activated by two cytokines, IL-1 alpha and IL-1 beta. To date, IL-1 beta has been the most studied, and therefore better understood of the two cytokines and has thus been the focus of many drug development efforts. By contrast, IL-1 alpha is less well understood, however, there have been studies that demonstrate expression of IL-1 alpha is activated by environmental stress, such as pathogens, and is directly linked to the inflammasome, which is responsible for activation of inflammatory processes (Fettelschoss A et al., (2011) Inflammasome activation and IL-1 beta target IL-1 alpha for secretion as opposed to surface expression, PNAS 108(44): 18055-18060).

IL-1 alpha is known to be involved in regulating inflammation in diverse conditions, including acne (Tanghetti E (2013) The Role of Inflammation in the pathology of acne. Journal of Clinical Aesthetic Dermatology 6(9):27-35), and hair loss/alopecia (Harmon C S and Nevins T D (1993) IL-1 alpha inhibits human hair follicle growth and hair fiber production in whole-organ cultures Lymphokine Cytokine Res 12(4):197-203). IL-1 alpha also plays a role in blemishes or skin darkening (Hirobe T & Ootaka H (2007) Interleukin-1α Stimulates the Differentiation of Melanocytes but Inhibits the Proliferation of Melanoblasts from Neonatal Mouse Epidermis. Zoological Science, 24(10):959-970), autoimmune skin conditions such as eczema, psoriasis, lupus erythematosus (Jensen L E (2010) Targeting theIL-1 family members in skin inflammation. Curr Opinion Investig Drugs 11(11):1211-1220), non-alcoholic steatohepatitis (NASH), atherosclerosis (Tilg H et al., (2016) Interleukin-1 and Inflammasomes in Alcoholic Liver Disease/Acute Alcoholic Hepatitis and Nonalcoholic Fatty Liver Disease/Nonalcoholic Steatohepatitis. Hepatology 64(3):955-965), atherosclerosis (Freigang, S. et al., Fatty acid-induced mitochondrial uncoupling elicits inflammasome-independent IL-1alpha and sterile vascular inflammation in atherosclerosis. Nat. Immunol. 14, 1045-1053 (2013)) and cancer (Dinarello C. (2014). Interleukin-1α neutralisation in patients with cancer. The Lancet Oncology, 552-553). For example, Propionibacterium acnes is known to stimulate the production of IL-1 alpha, with acne comedones containing high levels of IL-1 alpha and that IL-1 alpha is involved in the development of the pilosebaceous unit (Tanghetti E (2013)). The development of antibodies against IL-1 alpha is one approach currently underway as an option for the treatment of acne (Carrasco et al., “An Open Label Phase 2 Study of MABp1 Monotherapy for the Treatment of Acne Vulgaris and Psychiatric Comorbidity”. J Drugs Dermatol. June 2015;14(6):560-564). Development of antibodies against IL-1 alpha for use in the treatment of cancer (Dinarello, C. (2014). Interleukin-1α neutralisation in patients with cancer. The Lancet Oncology, 552-553) is also currently underway

Other inflammatory conditions which could benefit from controlling the activity of IL-1 alpha include diseases involving granulomas, such as in-grown nails.

In the applicant's earlier application, published as WO 2006/047830, an extract from physiologically-stressed Arthrospira, for topical treatment of fungal and bacterial infections or infestations, was disclosed. The present inventors have surprisingly found that an extract from physiologically-stressed Arthrospira, can be used to treat diseases or conditions where there is no microbial infection or infestation.

In particular, the present inventors have found that an extract from physiologically-stressed Arthrospira, has the ability to regulate IL-1 alpha expression, and can therefore be used in the treatment of diseases and/or conditions having an inflammatory basis.

The reference to any prior art in this specification is not, and should not be taken as an acknowledgement or any form of suggestion that the prior art forms part of the common general knowledge in the art in Australia or in any other country.

### SUMMARY OF INVENTION

The present invention is directed to a biological extract having anti-inflammatory activity and/or the ability to stimulate growth of new cells. The invention is also directed to compositions comprising a biological extract having anti-inflammatory activity and/or the ability to stimulate growth of new cells, and use of such compositions in the treatment of diseases or conditions having an inflammatory basis and/or in the treatment of diseases or conditions of skin.

In a first aspect, the present invention provides a biological extract having anti-inflammatory activity.

In a second aspect, the invention provides a biological extract having inhibitory activity against IL-1 alpha.

In a third aspect, the invention provides a biological extract having the ability to stimulate growth of new cells.

In a fourth aspect, the invention provides a biological extract having anti-inflammatory activity and the ability to stimulate growth of new cells.

In a fifth aspect, the invention provides a pharmaceutical composition comprising a biological extract having anti-inflammatory activity, and a pharmaceutically acceptable carrier, solvent, base or excipient.

In a sixth aspect, the invention provides a pharmaceutical composition comprising biological extract having inhibitory activity against IL-1 alpha, and a pharmaceutically acceptable carrier, solvent, base or excipient.

In a seventh aspect, the invention provides a pharmaceutical composition comprising a biological extract having the ability to stimulate growth of new cells, and a pharmaceutically acceptable carrier, solvent, base or excipient.

In an eighth aspect, the invention provides a pharmaceutical composition comprising a biological extract having anti-inflammatory activity and the ability to stimulate growth of new cells, and a pharmaceutically acceptable carrier, solvent, base or excipient.

In a ninth aspect, the invention provides a method of treating an inflammatory disorder in a subject, comprising the step of administering to the subject a therapeutically effective amount of a biological extract provided by the first, second or third aspects, or a pharmaceutical composition provided by the fifth, sixth or seventh aspects.

In a tenth aspect, the invention provides a method of treating a dermatological condition in a subject where there is no microbial infection or infestation, comprising the step of administering to the subject a therapeutically effective amount of a biological extract provided by the first, second or third aspects, or a pharmaceutical composition provided by the fifth, sixth or seventh aspects.

In an eleventh aspect, the invention provides a method of treating an inflammatory disorder and a dermatological condition in a subject where there is no microbial infection or infestation, comprising the step of administering to the subject a therapeutically effective amount of a biological extract provided by the fourth aspect, or a pharmaceutical composition provided by eighth aspect.

In a twelfth aspect, the invention provides a therapeutically effective amount of a biological extract provided by the first, second or third aspects, or a pharmaceutical composition provided by the fifth, sixth or seventh aspects for treating an inflammatory disorder.

In a thirteenth aspect, the invention provides a therapeutically effective amount of a biological extract provided by the first, second or third aspects, or a pharmaceutical composition provided by the fifth, sixth or seventh aspects for treating a dermatological condition where there is no microbial infection or infestation.

In a fourteenth aspect, the invention provides a therapeutically effective amount of a biological extract provided by fourth aspect, or a pharmaceutical composition provided by the eighth aspect for treating an inflammatory disorder and a dermatological condition where there is no microbial infection or infestation.

In a fifteenth aspect, the invention provides use of a therapeutically effective amount of a biological extract provided by the first, second or third aspects in the manufacture of a medicament for treating an inflammatory disorder.

In a sixteenth aspect, the invention provides use of a therapeutically effective amount of a biological extract provided by the first, second or third aspects in the manufacture of a medicament for treating a dermatological condition where there is no microbial infection or infestation.

In a seventeenth aspect, the invention provides use of a therapeutically effective amount of a biological extract provided by the fourth aspect in the manufacture of a medicament for treating an inflammatory disorder and a dermatological condition where there is no microbial infection or infestation.

In an eighteenth aspect, the invention provides a method of screening a biological extract for anti-inflammatory activity, the method comprising assaying the IL-1 alpha inhibitory activity of the biological extract, wherein IL-1 alpha inhibitory activity is indicative of anti-inflammatory activity.

Throughout this specification, unless the context requires otherwise, the words “comprise”, “comprises” and “comprising” will be understood to imply the inclusion of a stated integer or group of integers but not the exclusion of any other integer or group of integers.

Any of the features described herein can be combined in any combination with any one or more of the other features described herein within the scope of the invention.

### BRIEF DESCRIPTION OF DRAWINGS

FIG. 1 is a graph showing IL-1 alpha production from a model of artificial human skin following 6 hours exposure to products comprising physiologically stressed Arthrospira (AMYCOT®).

FIG. 2 is a graph showing inhibition of surfactant-induced IL-1 alpha production in a model of artificial human skin by products comprising physiologically stressed Arthrospira (AMYCOT®).

FIG. 3 is a graph showing cell proliferation in transformed human keratinocytes (HaCaT cells) after treatment with a product comprising 18% physiologically stressed Arthrospira (AMYCOT®) at varying time periods.

FIG. 4 is a graph showing cell proliferation in HaCaT cells after treatment with a product comprising 8% physiologically stressed Arthrospira (AMYCOT®) at varying time periods.

FIG. 5 is a graph showing total protein content in HaCaT cells treated with varying concentrations of a product comprising 18% physiologically stressed Arthrospira (AMYCOT®) and untreated cells following various time periods.

FIG. 6 is a graph showing the percentage increase in total protein content in HaCaT cells treated with varying concentrations of a product comprising 18% physiologically stressed Arthrospira (AMYCOT®) compared to untreated cells at various time periods.

FIG. 7 is a graph showing total protein content in HaCaT cells treated with varying concentrations of a product comprising 8% physiologically stressed Arthrospira (AMYCOT®) and untreated cells following various time periods.

FIG. 8 is a graph showing the percentage increase in total protein content in HaCaT cells treated with varying concentrations of a product comprising 8% physiologically stressed Arthrospira (AMYCOT®) compared to untreated cells at various time periods.

FIG. 9A is an image of a granuloma prior to treatment with a topical product comprising 8% physiologically stressed Arthrospira.

FIG. 9B is an image of the granuloma of FIG. 9A after treatment with a topical product comprising 8% physiologically stressed Arthrospira.

FIGS. 10A and 10B are images of subjects suffering from acne prior to treatment with a topical product comprising 12% physiologically stressed Arthrospira.

FIGS. 10C and 10D are images of the subjects of FIGS. 10A and 10B, respectively, after four weeks of topical treatment with a product comprising 12% physiologically stressed Arthrospira.

FIG. 11 is a series of images of the right hand of a subject suffering from eczema (pompholyx). The images were taken before treatment and at days 1, 3, 5, 7 and 10 during treatment with a topical product comprising 12% physiologically stressed Arthrospira.

FIG. 12A is an image of a skin blemish prior to treatment with a topical product comprising 12% physiologically stressed Arthrospira.

FIG. 12B is an image of the skin blemish of FIG. 12A after two weeks of daily treatment with a topical product comprising 12% physiologically stressed Arthrospira.

### DESCRIPTION OF EMBODIMENTS

### Abbreviations

The following abbreviations are used throughout:
DMEM=Dulbecco's Modified Eagle's Medium
FCS=Fetal Calf Serum
HaCaT=transformed human keratinocytes
IL-1 alpha=interleukin-1 alpha
IL-1 beta=interleukin-1 beta
IL-1R=interleukin-1 receptor
MTT=3-[4,5-dimethylthiazol-2-yl]-2,5-diphenyl tetrazolium bromide
NASH=non-alcoholic steatohepatitis
OD=optical density
PBS=phosphate buffered saline
SLS=sodium lauryl sulfate

The present invention is predicated in part on the discovery that combinations of molecules in biological extracts have inhibitory activity on IL-1 alpha and so can be used in the treatment of diseases or conditions having an inflammatory basis. The extracts can be used in the treatment of systemic diseases or conditions, including NASH, or skin diseases or conditions, including acne (including acne not resulting from Propionibacterium acnes), scarring, blemishes, eczema and the effects of ageing. Furthermore, the combinations of molecules in biological extracts stimulate cell growth leading to tissue regeneration and can therefore be used to simultaneously treat inflammatory diseases or conditions and stimulate tissue regeneration. The simultaneous effect is particularly advantageous for treating conditions such as acne (including acne not resulting from Propionibacterium acnes), eczema, alopecia or hair loss and NASH which have an inflammatory component and therefore benefit from anti-inflammatory activity, but also have a component that would benefit from tissue regeneration.

Accordingly, in a first aspect, the invention provides a biological extract having anti-inflammatory activity.

The term “biological extract” is used herein to refer to a sample obtained from a subject, wherein the subject is an animal, plant, fungi or bacteria. The biological extract can be a crude extract, a partially purified extract, substantially purified or purified. Where the biological extract is a crude extract, it can contain multiple active components, where an active component is a component that has anti-inflammatory activity and/or the ability to stimulate growth of new cells.

With each level of purification from the crude extract through to a purified extract, the number of active components in the extract is reduced. The active components can be selected from one or more of the group comprising proteins, peptides, small organic molecules and fatty acids. Preferably, the purified biological extract has at least two active components. Even more preferably, the purified biological extract comprises an active component having anti-inflammatory activity and an active component having the ability to stimulate growth of new cells, thus providing an extract having a combinatorial effect.

Preferably, the biological extract is obtained from a plant, fungi or bacteria. In a particularly preferred embodiment, the biological extract is obtained from one or more cyanobacteria from the genus, Arthrospira. That is the biological extract can be obtained from any suitable species of Arthrospira, including but not limited to, Arthrospira maxima. Alternatively, the biological extract can be from a mixture of species of Arthrospira.

In a preferred embodiment, the biological extract is prepared from physiologically stressed A. maxima.

Preferably, a crude extract of physiologically stressed A. maxima is subjected to at least one purification stage to provide a partially purified extract, a substantially purified extract or a purified extract. The at least one purification stage preferably results in an increase in the relative amino acid content of at least alanine, when compared to the amino acid content of the crude extract. Preferably, the alanine content is increased by 3-4 mole % as a result of the at least one purification stage. Even more preferably, the relative amino acid content of one or more amino acids selected from the group consisting of alanine, cysteine, glutamic acid/glutamine, glycine, aspartic acid/asparagine, proline, leucine, valine, isoleucine, serine, and phenylalanine, is increased as a result of the purification. The content of each of these amino acids is preferably independently increased by any value within the range 0.2-4 mole % as a result of the at least one purification stage.

The purification stage preferably results in a decrease in the relative amino acid content of at least lysine and tryptophan, when compared to the amino acid content of the crude extract. Preferably, the lysine content is decreased by 2-3 mole %, and the tryptophan content is also decreased by 2-3 mole % as a result of the at least one purification stage. Even more preferably, the relative amino acid content of one or more amino acids selected from the group consisting of lysine, tryptophan, methionine, tyrosine, and histidine is decreased as a result of the purification. The content of each of these amino acids is preferably independently decreased by any value within the range 0.3-3 mole % as a result of the at least one purification stage.

The purification stage preferably has an effect on the fatty acid content of the extract. In particular, the percentage of some fatty acids will increase, whilst the percentage of others will decrease when compared to the fatty acid content of the crude extract. Preferably, the purification stage results in an increase in the percentage of one or more fatty acids selected from the group consisting of palmitic acid, palmitoleic acid and stearic acid, when compared to the percentage of those fatty acids in the crude extract. Alternatively, the purification stage can result in a decrease in the percentage of one or more fatty acids selected from the group consisting of oleic acid, linolenic acid and gamma linolenic acid, when compared to the percentage of those fatty acids in the crude extract.

In a particularly preferred embodiment, the purification stage results in an increase in the percentage of one or more fatty acids selected from the group consisting of palmitic acid, palmitoleic acid and stearic acid, when compared to the percentage of those fatty acids in the crude extract, and a decrease in the percentage of one or more fatty acids selected from the group consisting of oleic acid, linolenic acid and gamma linolenic acid, when compared to the percentage of those fatty acids in the crude extract.

In a specific embodiment, the purification stage results in a 20-25% increase in the amount of palmitic acid in the extract, a 1-2% increase in the amount of palmitoleic acid, and a 0.1-1% increase in the amount of stearic acid.

In a further specific embodiment, the purification stage additionally results in a 12-16% decrease in the amount of gamma linolenic acid in the extract, an 8-12% decrease in the amount of linolenic acid, and a 1-2% decrease in the amount of oleic acid.

The term “anti-inflammatory activity” is used herein to refer to the property of reducing inflammation such as inflammation resulting from the effects of IL-1 alpha. For example, the anti-inflammatory activity can be reduction of inflammation associated with a granuloma. Alternatively, the anti-inflammatory activity can be reduction of inflammation associated with a skin disease or condition, including acne (including acne not resulting from Propionibacterium acnes), scarring and eczema. In a further alternative, the anti-inflammatory activity can be reduction of inflammation associated with NASH. Skin-aging and alopecia/hair loss are also conditions that result from an over-production of IL-1 alpha and therefore anti-inflammatory activity can also encompass reduction of inflammation associated with generation of free radicals (that can cause skin-aging) and inflammation associated with hair follicles.

In a second aspect, the invention provides a biological extract having inhibitory activity against IL-1 alpha.

As used herein, the term “inhibitory activity against IL-1 alpha” means reducing the release or production of IL-1 alpha. As IL-1 alpha plays a role in inflammation, reducing the release or production of IL-1 alpha will result in a reduction of inflammation.

In a third aspect, the invention provides a biological extract having the ability to stimulate growth of new cells.

As used herein, the term “stimulate growth of new cells” means stimulation that leads to tissue regeneration. Stimulation that leads to tissue regeneration is advantageous for the treatment of skin diseases or conditions such as acne (including acne not resulting from Propionibacterium acnes) and eczema. Tissue regeneration is also beneficial in the treatment of hair loss and/or alopecia. Further diseases or conditions that benefit from tissue regeneration include NASH. In particular, the term is used in the context of treatment of diseases or conditions that benefit from replenishment of cells.

The new cells can therefore be any type of mammalian cell. Preferably, the new cells are selected from the group consisting of skin cells. hair follicle cells and liver cells. In a particularly preferred embodiment, the new cells are skin cells.

In a fourth aspect, the invention provides a biological extract having anti-inflammatory activity and the ability to stimulate growth of new cells.

Preferably, the anti-inflammatory activity acts in combination with the ability to stimulate growth of new cells. For example, skin diseases or conditions such as acne (including acne not resulting from Propionibacterium acnes) and eczema which have an inflammatory component and also result in cell damage (such as scarring or fibrosis) would benefit from a combination of anti-inflammatory activity and stimulation of the growth of new cells.

In a fifth aspect, the invention provides a pharmaceutical composition comprising a biological extract having anti-inflammatory activity, and a pharmaceutically acceptable carrier, solvent, base or excipient.

The phrase “pharmaceutical composition” as used herein encompasses pharmaceutical and veterinary compositions.

The concentration of the biological extract in the pharmaceutical composition can vary depending on the application. For example, the concentration of the biological extract in the pharmaceutical composition can be any value between about 0.01% and about 100%. Preferably, the concentration of the biological extract in the pharmaceutical composition is between about 5% and about 20%. The concentration of the biological extract in the pharmaceutical composition can thus be about 5, 6, 7, 8, 9, 10, 11, 12, 13, 14, 15, 16, 17, 18, 19 or about 20%.

The pharmaceutical composition can be administered in any suitable form. For example, the pharmaceutical composition can be in a form suitable for oral administration. Suitable forms for oral administration include as a solid, such as capsules, tablets, pills, powders and granules. Suitable forms for oral administration also include as a liquid, such as emulsions, microemulsions, solutions, suspensions, syrups and elixirs.

In alternative embodiments, the pharmaceutical composition can be in a form suitable for topical or transdermal administration. Suitable forms for topical or transdermal administration include ointments, pastes, creams, lotions, gels, powders, solutions, sprays, inhalants and patches.

In a ninth aspect, the invention provides a method of treating an inflammatory disorder in a subject, comprising the step of administering to the subject a therapeutically effective amount of a biological extract provided by the first, second or third aspects, or a pharmaceutical composition provided by the fifth, sixth or seventh aspects.

The subject for treatment can be a human, mammal or animal. Preferably, the subject is a human or other type of mammal.

A “therapeutically effective amount” is the amount effective for treating an inflammatory disorder. The inflammatory disorder can be any disease or condition that has an inflammatory basis. The inflammatory disorder can therefore be a disease or condition of the skin, such as acne (including acne not resulting from Propionibacterium acnes), eczema. Alternatively, the inflammatory disorder could be a disease or condition that involves granulomas, such as in-grown nails. In a further alternative, the inflammatory disease or condition could be NASH.

The step of administering can be undertaken by any suitable means. Preferably, the therapeutically effective amount of the biological extract is administered orally or topically.

In a tenth aspect, the invention provides a method of treating a dermatological condition in a subject where there is no microbial infection or infestation, comprising the step of administering to the subject a therapeutically effective amount of a biological extract provided by the first, second or third aspects, or a pharmaceutical composition provided by the fifth, sixth or seventh aspects.

The dermatological condition can be any disease or condition of the skin where there is no microbial infection or infestation. Preferably, the dermatological condition has an inflammatory basis. For example, the dermatological condition can be acne (such as acne not resulting from Propionibacterium acnes), eczema, pigmentation, blemishes, scarring, granuloma or ageing.

It will be appreciated that the following examples have been provided for the purpose of illustrating preferred embodiments of the present invention. Therefore, it would be understood that the present invention should not be considered to be limited solely to the features as described in the examples.

### EXAMPLES

### Example 1

### In Vitro Evaluation of the Anti-Inflammatory Activity of Products Comprising Physiologically Stressed Arthrospira (AMYCOT®)

A reconstructed artificial human skin model comprising normal human epidermal keratinocytes, growing as an integrated three-dimensional cell culture model (Skinethic, Nice, France) was ustilised as an in vitro mimic of human skin. The model exhibits normal barrier functions, due to the presence of a differential stratum corneum). Normal human epidermal keratinocytes were seeded on a collagen matrix and grown in a serum-free medium to reach a multilayer conformation with a differentiated stratum corneum at the surface. Epidermis units having a 0.5 cm² diameter were purchased directly from Skinethic at the 16^{th} day of culture. The epidermis layer was placed in a transwell chamber, on a porous membrane. Undiluted duplicate samples of the products to be tested (10-15 mg) were applied on the upper keratinised layer of skin, with or without pre-treatment with 0.5% SLS (30 minutes). After 16 hours at 37° C. under 5% CO₂, the samples were removed and the skin washed twice with PBS. An MTT assay was then undertaken to evaluate cell survival and therefore to assess irritation potential of the samples.

The two samples tested were a dermaceutical cream of 18% physiologically stressed Arthrospira (‘Amycot 18%’) and a dermaceutical lotion of 8% physiologically stressed Arthrospira (‘Amycot 8%’).

The key component of the MTT assay, is (3-[4,5-dimethylthiazol-2-yl]-2,5-diphenyl tetrazolium bromide (MTT) which is yellow-coloured in solution. Mitochondrial dehydrogenases of viable cells cleave the tetrazolium ring, leading to the formation of purple crystals which are insoluble in aqueous solutions. The crystals are re-dissolved in acidified isopropanol and the resulting purple solution is measured spectrophotometrically. An increase or decrease in cell number results in a concomitant change in the amount of formazan formed, indicating the degree of cytotoxicity caused by the test material.

In the present assays, after exposure of the cells to the test material, the cells were washed with Dulbecco's PBS. After removal of the PBS, the MTT-medium was added to each culture well and the cells were incubated at 37° C. for four hours. Following incubation, the MTT-medium was removed and an MTT solubilisation solution (acidified isopropanol) was added to each well. The culture plate was shaken on a gyratory plate shaker for 20-30 minutes, ensuring that all the crystals had dissolved and formed a homogenous solution. The absorbance of each solution was measured on a microplate reader, with background subtraction. The results are shown in Table 1, and are expressed in terms of viability according to Equation 1:

% viability=OD treated cultures×100/OD untreated control cultures (Equation 1)

TABLE 1
% cell survival on 3D epidermis after 16
hours of exposure vs untreated control

| Sample | % viability |
|---|---|
| Amycot 18% | 87.6 |
| Amycot 18% + SLS 0.5% | 3.5 |
| Amycot 8% | 86.9 |
| Amycot 8% + SLS 0.5% | 3.2 |
| SLS 0.5% | 3.6 |
| Negative control | 100 |

The data indicates that after exposure to SLS (0.5%) for 16 hours, there is a 95% decrease in cell survival. The test compounds without SLS do not show cell mortality at 16 hours. The results are therefore indicative of good biocompatibility of the samples with skin, and furthermore that there is an absence of a cytotoxic and/or irritating effect on skin from the samples.

Inhibition of the release of IL-1 alpha was measured as an indicator of skin irritation, using commercially available ELISA test kits. A standard plot designed with the cytokine IL-1 alpha allows the titration in the cell medium by reading the absorbance at defined wavelengths, after specific identification with colorimetric reactions based on antibodies. For this assay, 200 μL of culture medium in the lower compartment of the well was collected at 6 hours and 16 hours. A solution of SLS (0.5%) was used as the positive control.

The results are shown in Table 2.

TABLE 2
IL-1 alpha release from 3D human epidermis
after exposure to test samples

IL-1 alpha (pg/mL)

| Sample | 6 hours | 16 hours |
|---|---|---|
| Amycot 18% | 27.8 | 35.6 |
| Amycot 18% + SLS 0.5% | 49.99 | 212.7 |
| Amycot 8% | 38.7 | 45.1 |
| Amycot 8% + SLS 0.5% | 39.81 | 264.4 |
| SLS 0.5% | 71.5 | 179.8 |
| Negative control | 15.16 | 15.2 |

The results following exposure to the samples for 6 hours are also shown in FIG. 1.

Inhibition of SLS-induced IL-1 alpha release by the tested products was also tested, with the results set out in Table 3.

TABLE 3
inhibition of SLS-induced IL-1 alpha after
treatment with the tested products.

| Sample | 6 hours | 16 hours |
|---|---|---|
| Amycot 18% | 38.18 | no inhibition |
| Amycot 8% | 56.25 | no inhibition |

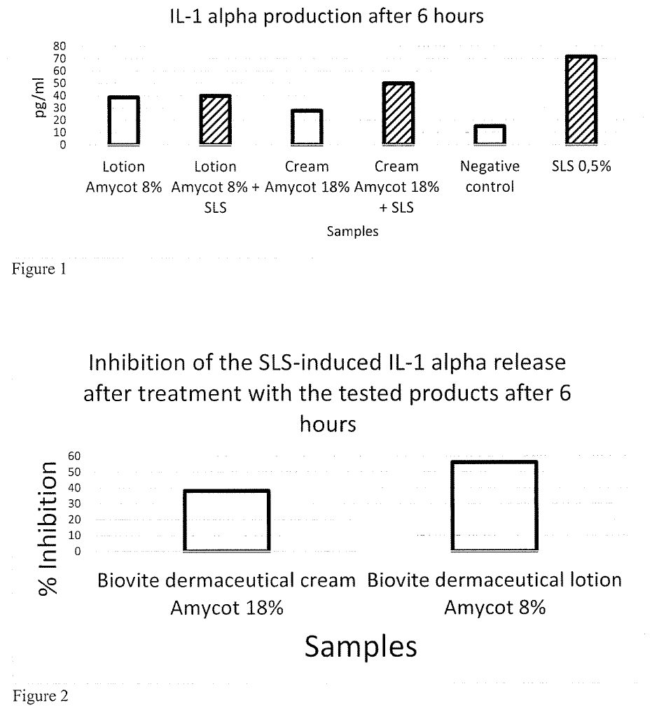

The results showing inhibition of SLS-induced IL-1 alpha release 6 hours after treatment with the tested products are also depicted in FIG. 2.

The results indicate that both Amycot 18% and Amycot 8% are able to inhibit the SLS-induced release of IL-1 alpha, 6 hours after treatment, however, this effect disappears 16 hours after treatment. Both products do cause a sight increase in IL-1 alpha release in untreated cells, but this increase is far lower than that resulting from the irritant effect of 0.5% SLS.

### Example 2

### In Vitro Evaluation of the Effect of Products Comprising Physiologically Stressed Arthrospira (AMYCOT®) on Cell Proliferation and Protein Synthesis

A cell survival assay using cultured keratinocytes was utilised to determine cell proliferation following exposure to the test samples. An MTT assay was utilised in the cell survival assay. In addition, a total cell protein content determination in keratinocytes was undertaken following exposure to serial dilutions of the test samples at different endpoints, utilising a Bradford test. The two samples tested were a dermaceutical cream of 18% physiologically stressed Arthrospira (‘Amycot 18%’) and a dermaceutical lotion of 8% physiologically stressed Arthrospira (‘Amycot 8%’).

A cell line established from adult human skin (HaCaT) was utilised. The cell line consists of transformed human keratinocytes and has full epidermal differentiation capacity.

HaCaT cells were seeded in 96 well plates (2,500 cells/well) for 24 hours in Dulbecco's Modified Eagle's Medium (DMEM) and 10% Fetal Calf Serum (FCS). Fresh medium was then added, supplement only with 5% FCS and with scalar dilutions of the two samples, Amycot 18% and Amycot 8%, dissolved in the medium. Non-treated cells were used as negative controls. For each dilution, two duplicate tests were performed, and repeated twice. After 24, 48 and 72 hours, the viability of the cells was tested using the MTT assay, and the total protein content was tested using the Bradford assay.

For the MTT assay, if cells are alive, mitochondrial dehydrogenases within the cells can cleave the tetrazolium ring of MTT, leading to the formation of purple crystals which are insoluble in aqueous solutions. The crystals are dissolved in acidified isopropanol and the resulting purple solution measured spectrophotometrically. After exposure of the HaCaT cells to the samples, the cells were washed with Dulbecco's PBS. MTT medium was added to each culture well and the cells incubated at 37° C. Following incubation, the MTT medium was removed and the cells treated with MTT solubilisation solution. The plate was then shaken on a gyratory plate shaker to ensure that all the crystals had dissolved from the cells and formed a homogenous solution. The absorbance was measured on a microplate reader, with background clearing. The results for Amycot 18% and Amycot 8% are shown in Tables 4 and 5, respectively, and are expressed in terms of viability according to Equation 1:

% viability=OD treated cultures×100/OD untreated control cultures (Equation 1)

TABLE 4
% cell viability for cells treated with Amycot 18%

| Concentration (mg/mL) | 24 hours | 48 hours | 72 hours |
|---|---|---|---|
| 0.1 | 55.0 | 85.5 | 96.0 |
| 0.01 | 106.6 | 84.2 | 101.9 |
| 0.001 | 128.8 | 99.8 | 95.0 |

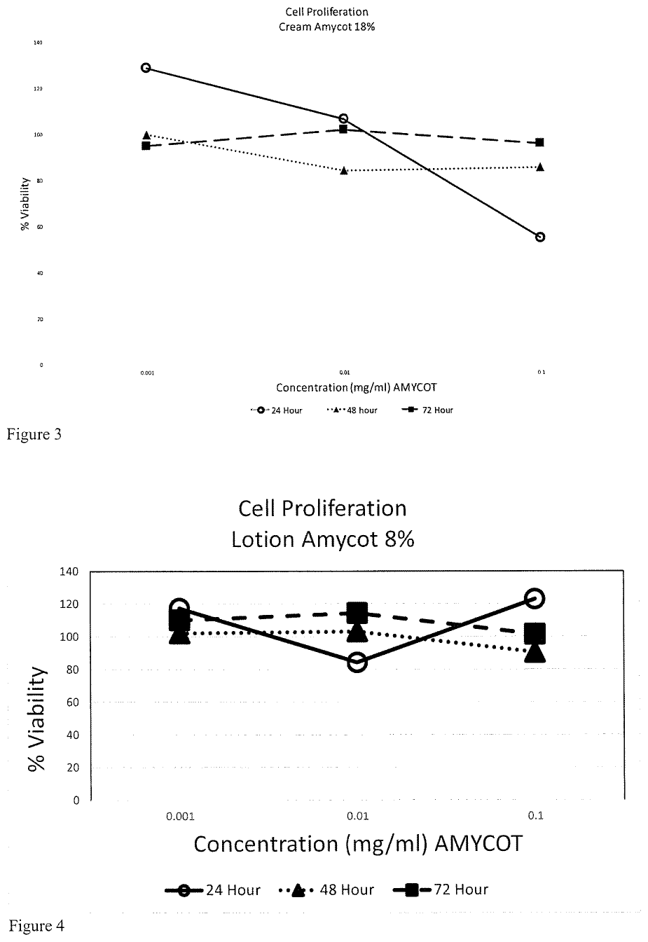

This data indicates there is an increase in cell proliferation compared to untreated cells following 24 hour exposure at 0.001 mg/mL. The results for cell proliferation following exposure to Amycot 18% are also depicted in FIG. 3.

TABLE 5
% cell viability for cells treated with Amycot 8%

| Concentration (mg/mL) | 24 hours | 48 hours | 72 hours |
|---|---|---|---|
| 0.1 | 122.9 | 90.5 | 101.5 |
| 0.01 | 84.2 | 103.1 | 114.4 |
| 0.001 | 117.4 | 102.2 | 110.1 |

This data indicates there is an increase in cell proliferation compared to untreated cells following 24 hours and 72 hours exposure at various concentrations. The highest activity is observed following 24 hours exposures at a concentration of 0.1 mg/mL. The results for cell proliferation following exposure to Amycot 8% are also depicted in FIG. 4.

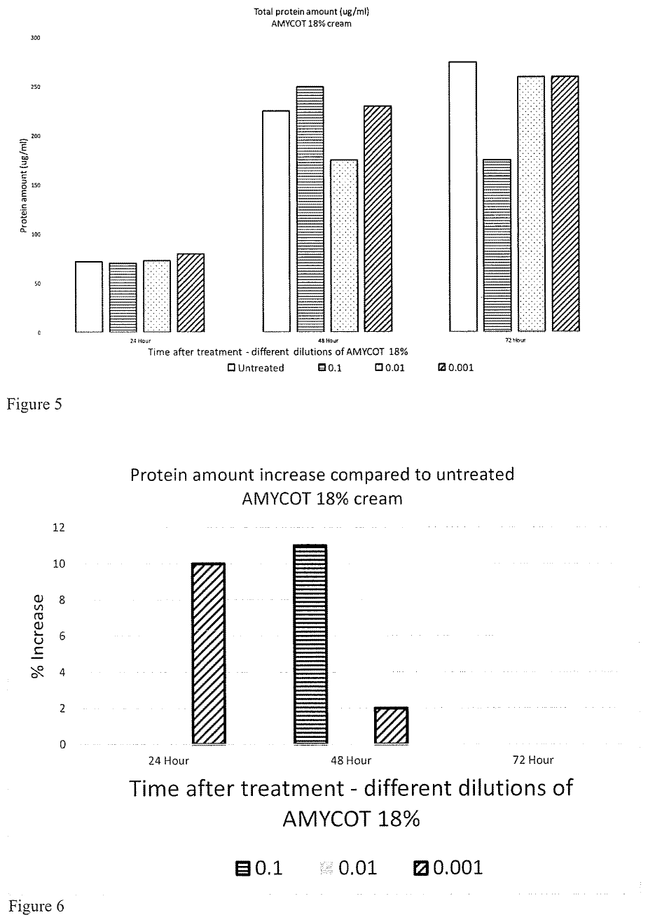

The Bradford assay for total protein content was performed using the Bio-Rad protein assay kit. Cells were lysed with 0.1 N NaOH, then diluted with Hank's Solution and then stained with a specific dye for proteins. Absorbance measurements were read at 595 nm, and protein titration was calculated on the basis of the measured optical density (OD) compared to a titration plot with albumin. The total protein for the same number of cells was plotted against the concentration of Amycot 18% and compared with the negative control (untreated cells), as shown in FIG. 5. The percentage increase compared to untreated cells is shown in FIG. 6.

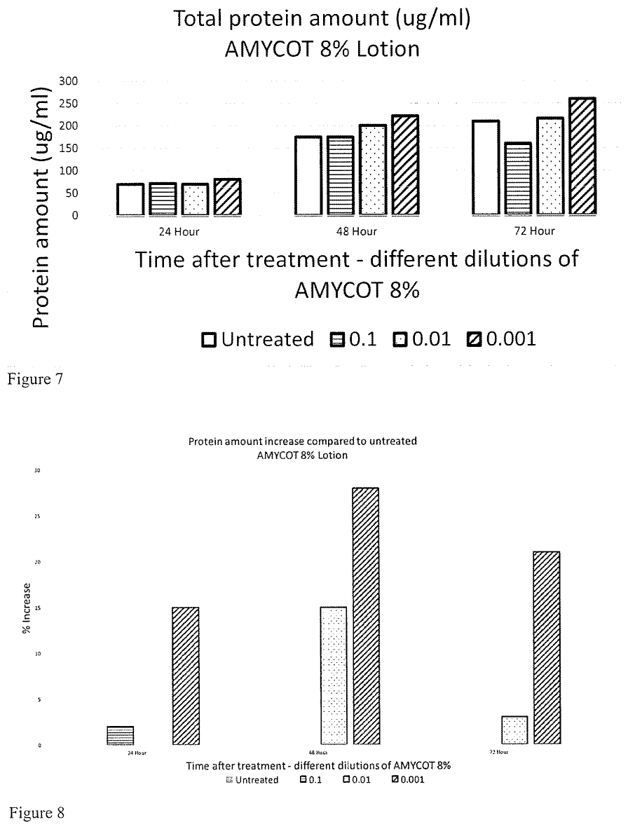

Corresponding data for cells treated with Amycot 8% are shown in FIGS. 7 and 8.

The data show an increase in protein synthesis compared to untreated cells for Amycot 18% at 0.1 mg/mL after 48 hours exposure, and at 0.001 mg/mL after 24 hours exposure. By contrast, use of Amycot 8% resulted in an increase in protein synthesis following 24, 48 and 72 hours exposures, particularly at a concentration of 0.001mg/mL.

In summary, the results show that Amycot 8% is effective in stimulating the growth of skin-derived cells such as keratinocytes after 24, 48 and 72 hours exposure, and that Amycot 18% increases cell proliferation after 24 hours exposure. Furthermore, whilst Amycot 8% and Amycot 18% result in a protein content increase, Amycot 8% is more effective than Amycot 18% in stimulating protein neosynthesis in keratinocytes, with the highest effect after 48 hours exposure at a concentration of 0.001 mg/mL.

### Example 3

### Anti-Inflammatory Activity of Product Comprising Physiologically Stressed Arthrospira (AMYCOT®)

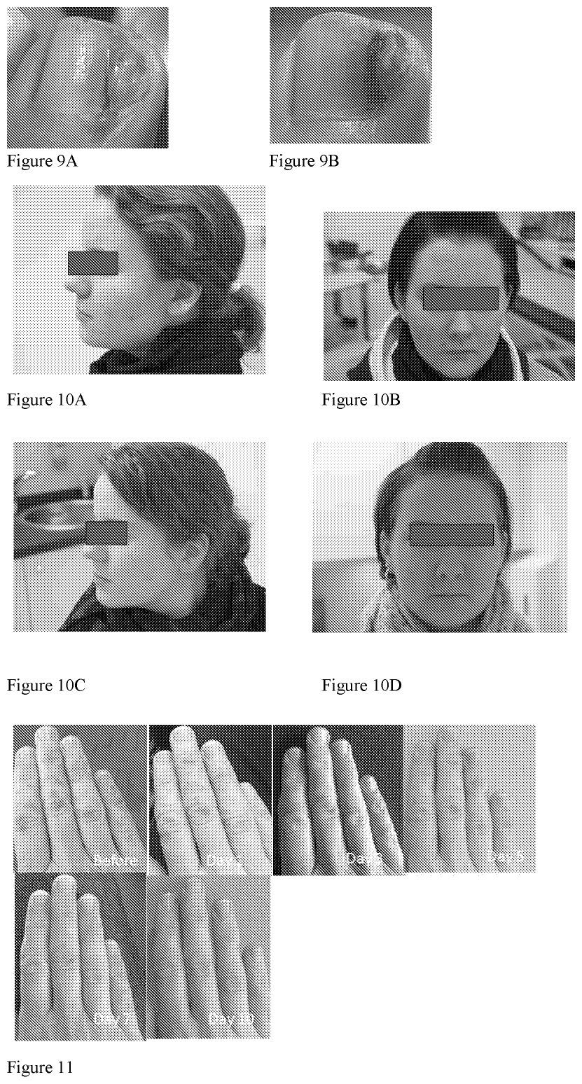

The anti-inflammatory activity of a product comprising 8% physiologically stressed Arthrospira (AMYCOT®) was tested on a granuloma arising from an in-grown nail. The subject was a female, aged 16 years, and the granuloma caused a great deal of pain. In FIG. 9A there is shown an image of the granuloma prior to treatment. The granuloma was treated with a topical product comprising 8% physiologically stressed Arthrospira. Within 12 hours of the initial treatment, the subject reported that the pain had subsided. The granuloma 24 hours after treatment is shown in FIG. 9B.

### Example 4

### Effect of Physiologically Stressed Arthrospira (AMYCOT®) on Acne

The effect of a product comprising 12% physiologically stressed Arthrospira (AMYCOT®) was tested on subjects having acne. In FIGS. 10A and 10B there are shown subjects suffering from acne. The comedones are quite pronounced and red, indicative of the presence of inflammation. A topical product comprising 12% physiologically stressed Arthrospira was applied to the affected area of each subject. The subjects are shown in FIGS. 10C and 10D after four weeks of topical treatment. The comedones are less pronounced, with reduced redness, indicating the topical product had an anti-inflammatory effect.

### Example 5

### Effect of Physiologically Stressed Arthrospira (AMYCOT®) on Eczema (Pompholyx)

The effect of a product comprising 12% physiologically stressed Arthrospira (AMYCOT®) was tested on a subject having eczema (pompholyx). The subject was a female, aged 42 years, and the eczema was red, itchy and had been present for 8 years. Commercially available eczema treatments had not been successful in clearing the eczema. The eczema was treated with a topical product comprising 12% physiologically stressed Arthrospira. In FIG. 11 there are shown a series of images of the right hand of the subject, from before treatment and at days 1, 3, 5, 7 and 10 during treatment. Within 24 hours (day 1) of the initial treatment, the redness has reduced and by day 10, the eczema had disappeared, indicating that the topical product had an anti-inflammatory effect.

### Example 6

### Effect of Physiologically Stressed Arthrospira (AMYCOT®) on a Skin Blemish

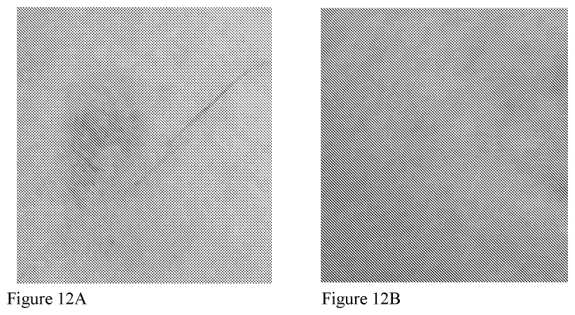

The effect of a product comprising 12% physiologically stressed Arthrospira (AMYCOT®) was tested on a skin blemish of a subject. The subject was a female, aged 55 years. In FIG. 12A there is shown an image of the skin blemish prior to treatment. A topical product comprising 12% physiologically stressed Arthrospira was applied daily to the skin blemish for two weeks. The area of the skin blemish after two weeks of daily treatment is shown in FIG. 12B. The skin blemish is no longer visible, indicating the topical product had an effect on the skin blemish, even though there was no microbial infection or infestation.

### Example 7

### Protein Analysis of Processed Arthrospira Maxima (AMYCOT®)

Processed Arthrospira maxima (AMYCOT®) was prepared as previously described in WO 2006/047830. The amount of AMYCOT® corresponded to at least 80% of the starting biomass of Arthrospira maxima.

The processed Arthrospira maxima was subjected to quantitative amino acid analysis, with cysteine analysis and tryptophan analysis undertaken separately. The unprocessed source Arthrospira maxima was also subjected to quantitative amino acid analysis, cysteine analysis and tryptophan analysis, for comparison with the processed Arthrospira maxima.

For the quantitative amino acid analysis, samples of processed and source Arthrospira maxima underwent liquid hydrolysis in 6M hydrochloric acid at 110° C. for 24 hours. Cysteine analysis was undertaken using performic acid oxidation followed by acid hydrolysis at 110° C. for 24 hours. Tryptophan analysis was undertaken using hydroxide hydrolysis at 110° C. for 24 hours.

Following hydrolysis, the amino acid content was analysed using AccQ-Tag chemistry (Waters Corporation). Samples were analysed in duplicate, and the results, expressed as an average, are set out in Tables 6 to 9.

TABLE 6
amino acid analysis of processed
Arthrospira maxima (AMYCOT ®), excluding
cysteine and tryptophan

| | Amino Acid | Amino Acid | |
|---|---|---|---|
| | (—H₂O) * | (free) ** | |
| Amino Acid | (mg/g of sample) | (mg/g of sample) | Mole % |
| Aspartic acid + | 37.4 | 43.3 | 10.3 |
| Asparagine | | | |
| Serine | 17.1 | 20.7 | 6.2 |
| Glutamic acid + | 51.9 | 59.2 | 12.7 |
| Glutamine | | | |
| Glycine | 17.5 | 23.1 | 9.7 |
| Histidine | 4.9 | 5.5 | 1.1 |
| Arginine | 27.1 | 30.2 | 5.4 |
| Threonine | 16.8 | 19.8 | 5.2 |
| Alanine | 32.1 | 40.2 | 14.2 |
| Proline | 13.5 | 16.0 | 4.4 |
| Tyrosine | 10.9 | 12.1 | 2.1 |
| Valine | 24.2 | 28.6 | 7.7 |
| Methionine | 1.3 | 1.5 | 0.3 |
| Lysine | 5.0 | 5.7 | 1.2 |
| Isoleucine | 22.6 | 26.2 | 6.3 |
| Leucine | 33.2 | 38.5 | 9.3 |
| Phenylalanine | 18.5 | 20.7 | 3.9 |
| Total | 334.1 | | 100.0 |

* calculation based on amino acid residue mass in protein (molecular weight minus H₂O)
** calculation based on free amino acid molecular weight

TABLE 7
amino acid analysis of processed
Arthrospira maxima (AMYCOT ®), including
cysteine and tryptophan

| | Amino Acid | Amino Acid | |
|---|---|---|---|
| | (—H₂O) * | (free) ** | |
| Amino Acid | (mg/g of sample) | (mg/g of sample) | Mole % |
| Aspartic acid + | 37.8 | 43.7 | 10.2 |
| Asparagine | | | |
| Serine | 16.8 | 20.2 | 6.0 |
| Glutamic acid + | 52.2 | 59.5 | 12.6 |
| Glutamine | | | |
| Glycine | 16.5 | 21.7 | 9.0 |
| Histidine | 4.2 | 4.8 | 1.0 |
| Arginine | 27.4 | 30.6 | 5.5 |
| Threonine | 17.4 | 20.5 | 5.4 |
| Alanine | 32.0 | 40.0 | 14.0 |
| Proline | 13.8 | 16.4 | 4.4 |
| Cysteine | 6.1 | 7.2 | 2.8 |
| Tyrosine | 10.1 | 11.3 | 1.9 |
| Valine | 22.6 | 26.7 | 7.1 |
| Methionine | 1.1 | 1.3 | 0.3 |
| Lysine | 5.1 | 5.8 | 1.2 |
| Isoleucine | 21.3 | 24.6 | 5.9 |
| Leucine | 32.0 | 37.1 | 8.8 |
| Phenylalanine | 16.4 | 18.4 | 3.5 |
| Tryptophan | 0.5 | 0.5 | 0.5 |
| Total | 333.1 | | 100.0 |

* calculation based on amino acid residue mass in protein (molecular weight minus H₂O)
** calculation based on free amino acid molecular weight

TABLE 8
amino acid analysis of source Arthrospira maxima,
excluding cysteine and tryptophan

| | Amino Acid | Amino Acid | |
|---|---|---|---|
| | (—H₂O) * | (free) ** | |
| Amino Acid | (mg/g of sample) | (mg/g of sample) | Mole % |
| Aspartic acid + | 40.7 | 47.0 | 10.3 |
| Asparagine | | | |
| Serine | 18.1 | 21.8 | 6.1 |
| Glutamic acid + | 56.3 | 64.1 | 12.7 |
| Glutamine | | | |
| Glycine | 17.6 | 23.1 | 9.0 |
| Histidine | 6.4 | 7.3 | 1.4 |
| Arginine | 32.9 | 36.7 | 6.1 |
| Threonine | 18.6 | 21.9 | 5.4 |
| Alanine | 27.5 | 34.4 | 11.3 |
| Proline | 14.7 | 17.4 | 4.4 |
| Tyrosine | 14.0 | 15.5 | 2.5 |
| Valine | 24.8 | 29.3 | 7.3 |
| Methionine | 4.2 | 4.8 | 0.9 |
| Lysine | 17.6 | 20.1 | 4.0 |
| Isoleucine | 23.4 | 27.1 | 6.0 |
| Leucine | 34.9 | 40.5 | 9.0 |
| Phenylalanine | 17.9 | 20.1 | 3.6 |
| Total | 369.6 | | 100.0 |

* calculation based on amino acid residue mass in protein (molecular weight minus H₂O)
** calculation based on free amino acid molecular weight

TABLE 9
amino acid analysis of source Arthrospira maxima,
including cysteine and tryptophan

| | Amino Acid | Amino Acid | |
|---|---|---|---|
| | (—H₂O) * | (free) ** | |
| Amino Acid | (mg/g of sample) | (mg/g of sample) | Mole % |
| Aspartic acid + | 41.2 | 47.7 | 9.8 |
| Asparagine | | | |
| Serine | 18.4 | 22.2 | 5.8 |
| Glutamic acid + | 57.0 | 64.9 | 12.1 |
| Glutamine | | | |
| Glycine | 17.6 | 23.1 | 8.5 |
| Histidine | 6.3 | 7.2 | 1.3 |
| Arginine | 34.6 | 38.6 | 6.1 |
| Threonine | 20.2 | 23.8 | 5.5 |
| Alanine | 27.8 | 34.8 | 10.7 |
| Proline | 14.1 | 16.7 | 4.0 |
| Cysteine | 6.9 | 8.2 | 2.1 |
| Tyrosine | 14.3 | 15.8 | 2.4 |
| Valine | 24.4 | 28.8 | 6.8 |
| Methionine | 5.1 | 5.8 | 1.1 |
| Lysine | 17.5 | 19.9 | 3.7 |
| Isoleucine | 23.0 | 26.6 | 5.6 |
| Leucine | 34.4 | 39.9 | 8.4 |
| Phenylalanine | 17.6 | 19.7 | 3.3 |
| Tryptophan | 3.9 | 4.3 | 2.9 |
| Total | 384.3 | | 100.0 |

* calculation based on amino acid residue mass in protein (molecular weight minus H₂O)
** calculation based on free amino acid molecular weight

For ease of reference, the mole % amino acid analysis results for the source Arthrospira maxima and processed Arthrospira maxima have been extracted from Tables 6 to 9, and are set out in Tables 10 and 11.

TABLE 10
amino acid analysis of source and processed
Arthrospira maxima, excluding cysteine and tryptophan

| Amino Acid | Mole % (source) | Mole % (processed) |
|---|---|---|
| Aspartic acid + | 10.3 | 10.3 |
| Asparagine | | |
| Serine | 6.1 | 6.2 |
| Glutamic acid + | 12.7 | 12.7 |
| Glutamine | | |
| Glycine | 9.0 | 9.7 |
| Histidine | 1.4 | 1.1 |
| Arginine | 6.1 | 5.4 |
| Threonine | 5.4 | 5.2 |
| Alanine | 11.3 | 14.2 |
| Proline | 4.4 | 4.4 |
| Tyrosine | 2.5 | 2.1 |
| Valine | 7.3 | 7.7 |
| Methionine | 0.9 | 0.3 |
| Lysine | 4.0 | 1.2 |
| Isoleucine | 6.0 | 6.3 |
| Leucine | 9.0 | 9.3 |
| Phenylalanine | 3.6 | 3.9 |
| Total | 100.0 | 100.0 |

TABLE 11
amino acid analysis of source and processed
Arthrospira maxima, including cysteine and tryptophan

| Amino Acid | Mole % (source) | Mole % (processed) |
|---|---|---|
| Aspartic acid + | 9.8 | 10.2 |
| Asparagine | | |
| Serine | 5.8 | 6.0 |
| Glutamic acid + | 12.1 | 12.6 |
| Glutamine | | |
| Glycine | 8.5 | 9.0 |
| Histidine | 1.3 | 1.0 |
| Arginine | 6.1 | 5.5 |
| Threonine | 5.5 | 5.4 |
| Alanine | 10.7 | 14.0 |
| Proline | 4.0 | 4.4 |
| Cysteine | 2.1 | 2.8 |
| Tyrosine | 2.4 | 1.9 |
| Valine | 6.8 | 7.1 |
| Methionine | 1.1 | 0.3 |
| Lysine | 3.7 | 1.2 |
| Isoleucine | 5.6 | 5.9 |
| Leucine | 8.4 | 8.8 |
| Phenylalanine | 3.3 | 3.5 |
| Tryptophan | 2.9 | 0.5 |
| Total | 100.0 | 100.0 |

The data indicate that the steps of processing the crude, source Arthrospira maxima, result in an increase in the relative amino acid content of alanine, valine, isoleucine, leucine glycine, proline, cysteine, aspartic acid/asparagine, glutamic acid/glutamine, serine and phenylalanine in the processed Arthrospira maxima, when compared to the relative amino acid content of the source Arthrospira maxima. Concurrently, there is seen a decrease in the relative amino acid content of lysine, arginine, histidine, tyrosine, tryptophan and methionine in the processed Arthrospira maxima, when compared to the relative amino acid content of the source Arthrospira maxima.

### Example 8

### Fatty Acid Analysis of Processed Arthrospira maxima (AMYCOT®)

The processed Arthrospira maxima was subjected to fatty acid analysis. The unprocessed source Arthrospira maxima was also subjected to fatty acid analysis for comparison with the processed Arthrospira maxima.

The fatty acid profile of the samples was determined using a DB-WAX column (Agilent Technologies) by gas chromatograph—flame ionization detector (GC-FID). The results are set out in Table 12.

TABLE 12
fatty acid analysis of processed Arthrospira maxima
(AMYCOT ®) and source Arthrospira maxima

| | | processed |
|---|---|---|
| | source | Arthrospira maxima |
| fatty add | Arthrospira maxima | (AMYCOT ®) |
| palmitic acid (%) | 48.2 | 69.9 |
| palmitoleic acid (%) | 2.8 | 4.3 |
| stearic acid (%) | 2.4 | 2.7 |
| oleic acid (%) | 2.6 | 1.9 |
| linoleic acid (%) | 17.9 | 8.2 |
| gamma linolenic acid (%) | 18.9 | 4.6 |
| alpha linolenic add (%) | <0.1 | <0.1 |

The data indicate that the steps of processing the crude, source Arthrospira maxima, result in an increase in the percentage of palmitic acid, palmitoleic acid and stearic acid, and a decrease in the percentage of oleic acid, linolenic acid and gamma linolenic acid in the processed Arthrospira maxima, when compared to the percentage of those fatty acids in the source Arthrospira maxima.

Reference throughout this specification to ‘one embodiment’ or ‘an embodiment’ means that a particular feature, structure, or characteristic described in connection with the embodiment is included in at least one embodiment of the present invention. Thus, the appearance of the phrases ‘in one embodiment’ or ‘in an embodiment’ in various places throughout this specification are not necessarily all referring to the same embodiment. Furthermore, the particular features, structures, or characteristics may be combined in any suitable manner in one or more combinations.

In compliance with the statute, the invention has been described in language more or less specific to structural or methodical features. It is to be understood that the invention is not limited to specific features shown or described since the means herein described comprises preferred forms of putting the invention into effect. The invention is, therefore, claimed in any of its forms or modifications within the proper scope of the appended claims (if any) appropriately interpreted by those skilled in the art.

1. An extract from physiologically stressed Arthrospira, when the extract has anti-inflammatory activity.

2. The extract of claim 1, wherein the anti-inflammatory activity is inhibitory activity against IL-1 alpha.

3. The extract of claim 2, further having the ability to stimulate growth of new cells.

4. A pharmaceutical composition comprising the extract of claim 1, and a pharmaceutically acceptable carrier, solvent, base or excipient.

5. A method of treating (i) an inflammatory disorder in a subject; (ii) a dermatological condition in a subject, wherein the dermatological condition is not the result of a microbial infection or infestation; or (iii) an inflammatory disorder and a dermatological condition in a subject, wherein the dermatological condition is not the result of a microbial infection or infestation, comprising the step of administering to the subject a therapeutically effective amount of an extract according to claim 1.

6. (canceled)

7. (canceled)

8. (canceled)

9. (canceled)

10. (canceled)

11. (canceled)

12. (canceled)

13. (canceled)

14. A method of screening an extract of physiologically stressed Arthrospira for anti-inflammatory activity, the method comprising assaying the IL-1 alpha inhibitory activity of the extract, wherein IL-1 alpha inhibitory activity is indicative of anti-inflammatory activity.
